SEQUENCE LISTING (25 sequences)

SEQ ID NO: 1-2 - skipped (2 entries)

SEQ ID NO: 3 - DNA, 63 bp, synthetic construct
  source 1..63: mol_type = other DNA
gactggagttcagacgtgtgctcttccgatcnnnannntnncttacctgaggagacggtgacc

SEQ ID NO: 4 - DNA, 63 bp, synthetic construct
  source 1..63: mol_type = other DNA
gactggagttcagacgtgtgctcttccgatcnncnnnannncttacctgaggagacggtgacc

SEQ ID NO: 5 - DNA, 56 bp, synthetic construct
  source 1..56: mol_type = other DNA
acactctttccctacacgacgctcttccgatcggcctcaggaaggtctcctgcaag

SEQ ID NO: 6 - DNA, 54 bp, synthetic construct
  source 1..54: mol_type = other DNA
acactctttccctacacgacgctcttccgatcgtctggtcctacgctggaaccc

SEQ ID NO: 7 - DNA, 56 bp, synthetic construct
  source 1..56: mol_type = other DNA
acactctttccctacacgacgctcttccgatcctgggggtccctgagactctcctg

SEQ ID NO: 8 - DNA, 58 bp, synthetic construct
  source 1..58: mol_type = other DNA
acactctttccctacacgacgctcttccgatccttcggagacccctgtccctcacctg

SEQ ID NO: 9 - DNA, 52 bp, synthetic construct
  source 1..52: mol_type = other DNA
acactctttccctacacgacgctcttccggggagtctctgaagatctcctgt

SEQ ID NO: 10 - DNA, 56 bp, synthetic construct
  source 1..56: mol_type = other DNA
acactctttccctacacgacgctcttccgattcgcagaccctctcactcacctgtg

SEQ ID NO: 11 - DNA, 51 bp, synthetic construct
  source 1..51: mol_type = other DNA
acactctttccctacacgacgctcttccgatctgtcagcccccagggaagg

SEQ ID NO: 12 - DNA, 54 bp, synthetic construct
  source 1..54: mol_type = other DNA
acactctttccctacacgacgctcttccgatcggtccgcccaggctccagggaa

SEQ ID NO: 13 - DNA, 51 bp, synthetic construct
  source 1..51: mol_type = other DNA
acactctttccctacacgacgctcttccgatctgcccccccccagggaagg

SEQ ID NO: 14 - DNA, 56 bp, synthetic construct
  source 1..56: mol_type = other DNA
acactctttccctacacgacgctcttccgatcgggtggcccagatgcccgggaagg

SEQ ID NO: 15 - DNA, 58 bp, synthetic construct
  source 1..58: mol_type = other DNA
acactctttccctacacgacgctcttccgatctggatcaggcagtcccccatcgagag

SEQ ID NO: 16 - DNA, 57 bp, synthetic construct
  source 1..57: mol_type = other DNA
acactctttccctacacgacgctcttccgatcttgggggcgacaggcccctggacaa

SEQ ID NO: 17 - DNA, 57 bp, synthetic construct
  source 1..57: mol_type = other DNA
acactctttccctacacgacgctcttccgatctggagctgagcagcctgagatctga

SEQ ID NO: 18 - DNA, 57 bp, synthetic construct
  source 1..57: mol_type = other DNA
acactctttccctacacgacgctcttccgatccatgaccaacatggacccctgtgga

SEQ ID NO: 19 - DNA, 57 bp, synthetic construct
  source 1..57: mol_type = other DNA
acactctttccctacacgacgctcttccgatctctgcaaatgaacagcctgagagcc

SEQ ID NO: 20 - DNA, 57 bp, synthetic construct
  source 1..57: mol_type = other DNA
acactctttccctacacgacgctcttccgatcgagctctgtggaccgccgggacacg

SEQ ID NO: 21 - DNA, 57 bp, synthetic construct
  source 1..57: mol_type = other DNA
acactctttccctacacgacgctcttccgatccagcaccgcctacctgcagtggagc

SEQ ID NO: 22 - DNA, 57 bp, synthetic construct
  source 1..57: mol_type = other DNA
acactctttccctacacgacgctcttccgatcgttctccctgcagctgaactctgtg

SEQ ID NO: 23 - DNA, 57 bp, synthetic construct
  source 1..57: mol_type = other DNA
acactctttccctacacgacgctcttccgatcccagcacggcatatctgcagatcag

SEQ ID NO: 24 - DNA, 59 bp, synthetic construct
  source 1..59: mol_type = other DNA
gactggagttcagacgtggctcttccgatcnnnnnnnncttacctgaggagacgggacc

SEQ ID NO: 25 - DNA, 30 bp, synthetic construct
  source 1..30: mol_type = other DNA
gactggagttcagacgtggctcttccgatc

(19) United States
(12) Patent Application Publication
(10) Pub. No.: US 2024/0409924 A1
(43) Pub. Date: Dec. 12, 2024

(54) LOCKED NUCLEIC ACID-MODIFIED MOLECULAR BARCODE, REVERSE PRIMER, PRIMER SET, KIT, METHOD FOR INHIBITING NON-SPECIFIC AMPLIFICATION, AND USE THEREOF

(71) Applicant: BOE Technology Group Co., Ltd., Beijing (CN)

(72) Inventor: Shixue LI, Beijing (CN)

(21) Appl. No.: 18/702,323

(22) PCT Filed: May 17, 2023
(86) PCT No.: PCT/CN2023/094729

§ 371 Date: Apr. 17, 2024

(30) Foreign Application Priority Data
May 18, 2022 (CN) 202210551546.0

(51) Int. Cl. C12N 15/11; C12Q 1/6848; C12Q 1/6858; C12Q 1/6876

(52) U.S. Cl. CPC C12N 15/11; C12Q 1/6848; C12Q 1/6858; C12Q 1/6876; C12Q 2600/16

(57) ABSTRACT

Provided are a locked nucleic acid-modified molecular barcode, a reverse primer, a primer set, a kit, a method for inhibiting non-specific amplification, and use thereof. The total number Q of bases in the molecular barcode is greater than or equal to 10, and the number of degenerate bases N is greater than or equal to 8. The molecular barcode has m locked nucleic acid-modified sites, which divide the molecular barcode into m+1 segments, wherein the number of bases in each segment is 2 to 4, and m is greater than or equal to 2.

### CROSS-REFERENCE TO RELATED APPLICATIONS

The present application is a U.S. National Phase Entry of International PCT Application No. PCT/CN2023/094729 having an international filing date of May 17, 2023, which claims the priority of the Chinese Patent Application No. 202210551546.0, filed to CNIPA on May 18, 2022 and entitled “LOCKED NUCLEIC ACID-MODIFIED MOLECULAR BARCODE, REVERSE PRIMER, PRIMER SET, KIT, METHOD FOR INHIBITING NON-SPECIFIC AMPLIFICATION, AND USE THEREOF”. The contents of the above-identified applications are incorporated herein by reference in their entireties.

### REFERENCE TO SEQUENCE LISTING

The official copy of the Sequence Listing is submitted concurrently with the specification as an XML file, with a file name of “US2401866_Substitute Sequence Listing.xml”, a creation date of Apr. 17, 2024, and a size of 22 KB. The Sequence Listing is part of the specification and is incorporated in its entirety by reference herein.

### TECHNICAL FIELD

Embodiments of the present disclosure relate to, but are not limited to, the field of molecular biotechnology, in particular to a locked nucleic acid-modified molecular barcode, a reverse primer, a primer set, a kit, a method for inhibiting nonspecific amplification and use thereof.

### BACKGROUND

Immunoglobulin refers to globulin with antibody activity or chemical structure, which is a tetramer composed of two identical light chains and two identical heavy chains connected by disulfide bonds, wherein the light chain consists of variable region (V region), joining region (J region) and constant region (C region), and the heavy chain consists of variable region (V region), hinge region (D region), joining region (J region) and constant region (C region), which are encoded by corresponding V, D, J and C gene fragments respectively. There are a plurality of copies of V, D and J gene fragments in organisms, and during the development of B lymphocytes, the process of random combination of different V, D and J gene fragments to form functional immunoglobulins is called gene rearrangement.

Under normal physiological conditions, the immunoglobulin rearrangement sequences carried by B lymphocytes are relatively uniform, and there are little difference in the proportion of different sequences, showing “polyclonal rearrangement”. Lymphocytes lose normal function and proliferate malignantly in the process of lymphoma tumorigenesis, which process is accompanied by a great increase in the proportion of specific rearrangement sequences carried by the lymphocyte, showing “clonal rearrangement”. By detecting gene rearrangement in the lymphocyte and identifying “polyclonal rearrangement” or “clonal rearrangement” therein, whether a patient has normal lymph node enlargement or lymphoma can be determined.

### SUMMARY

The following is a summary of subject matters described herein in detail. This summary is not intended to limit the protection scope of claims.

The present disclosure provides a locked nucleic acid-modified molecular barcode, a reverse primer, a primer set, a kit, a method for inhibiting non-specific amplification and use thereof.

In a first aspect, embodiments of the present disclosure provide a locked nucleic acid-modified molecular barcode for inhibiting non-specific amplification in the process of gene rearrangement detection, wherein
the total number of bases in the molecular barcode, Q, is greater than or equal to 10, the molecular barcode has m locked nucleic acid-modified sites, which divide the molecular barcode into m+1 segments, wherein the number of bases in each segment is 2 to 4, and m is greater than or equal to 2.

Alternatively, m satisfies:

[Q/4]≤m≤[Q/3],
in the formula, Q denotes the total number of bases in the molecular barcode, [Q/3] denotes the maximum integer not exceeding Q/3, and [Q/4] denotes the maximum integer not exceeding Q/4.

Alternatively, the number of degenerate bases N is greater than or equal to 8.

Alternatively, the number of degenerate bases N is 8 to 10.

Alternatively, the number of bases in at least two segments in the molecular barcode is same.

Alternatively, the number of bases in two adjacent segments in the molecular barcode is same.

Alternatively, the total number of bases in the molecular barcode, Q, is 10, and the locked nucleic acid-modified sites comprise a position k site and a position k+4 site along the direction from the 5′ end to the 3′ end of the molecular barcode, wherein, k is greater than 2.

Alternatively, the locked nucleic acid-modified sites include a position 4 site and a position 8 site.

Alternatively, the sequence of the molecular barcode is set forth in SEQ ID NO. 1.

Alternatively, the locked nucleic acid-modified sites include a position 3 site and a position 7 site.

Alternatively, the sequence of the molecular barcode is set forth in SEQ ID NO. 2.

In a second aspect, one embodiment of the present disclosure provides a reverse primer, the reverse primer comprises the locked nucleic acid-modified molecular barcode of the first aspect, and the reverse primer comprises an adapter primer, a molecular barcode, and a specific primer.

Alternatively, the sequence of the reverse primer is set forth in SEQ ID NO. 3; or
the sequence of the reverse primer is set forth in SEQ ID NO. 4.

In a third aspect, another embodiment of the present disclosure provides a primer set comprising a forward primer and the reverse primer of the second aspect, and the forward primer comprises at least one of an FR1 forward primer, an FR2 forward primer, and an FR3 forward primer.

In a fourth aspect, yet another embodiment of the present disclosure provides a kit comprising:
DNA polymerase, buffer, and
the reverse primer of the second aspect.

In a fifth aspect, yet another embodiment of the present disclosure provides a method for inhibiting non-specific amplification in a gene rearrangement detection, which use the primer set of the third aspect to perform an amplification reaction, and the method comprises:
performing at least two rounds of PCR amplification reaction on the sample to be amplified to obtain an amplification product;
wherein, the primer used in a first round of PCR amplification reaction comprises the reverse primer, and the primer used in a second round of PCR amplification reaction comprises at least one of the FR1 forward primer, the FR2 forward primer and the FR3 forward primer.

Alternatively, in at least two rounds of the PCR amplification reaction, the primer used in the last round of the PCR amplification reaction is an adapter primer.

Alternatively, the concentration of the reverse primer is from 9 μmol/L to 11 μmol/L, and the added amount of the reverse primer is from 5 μL to 7 μL.

Alternatively, the total weight of DNA in the sample to be amplified is greater than or equal to 100 ng.

In a sixth aspect, yet another embodiment of the present disclosure provides a use of the locked nucleic acid-modified molecular barcode of the first aspect in preparing a reagent for gene rearrangement detection.

In the locked nucleic acid-modified molecular barcode provided by the embodiment of the present disclosure, by controlling the total number of bases in the molecular barcode, Q, to be greater more and equal to 10, controlling the number of the locked nucleic acid-modified sites to be greater more and equal to 2, controlling the number of bases in each segment in the molecular barcode, the locked nucleic acid-modified sites evenly separate the sequences of molecular barcodes. Because the base modified by the locked nucleic acid is a special bi-cyclic oligonucleotide derivative, the 2′-O, 4′-C sites of ribose form an oxamethylene bridge, a thiamethylene bridge or an azamethylene bridge through different water condensation actions to be connected to form a ring, and this ring bridge locks the N configuration of furanose C3′-endo type, reduces the flexibility of ribose or deoxyribose structure, and increases the stability of the local structure of phosphate backbone. And the base modified by the locked nucleic acid has the same phosphate backbone as DNA or RNA, so the recognition and binding ability of a base modified by the locked nucleic acid to a base on the target gene is stronger than that of a base not modified by the locked nucleic acid, that is to say, a locked nucleic acid modification is made in the molecular barcode of primers, so that the recognition and binding ability of the base in the primers modified by the locked nucleic acid to the base of the target gene is stronger than that of the complementary primer. The direct binding between the adapter primers of the amplification primers is avoided, thereby the adapter self-connection between the forward primers and the reverse primers can be inhibited, and the number of non-specific amplification products generated by the adapter self-connection between the forward primers and the reverse primers is reduced.

The above description is only an overview of technical schemes of the present disclosure, which may be implemented according to contents of the specification in order to be able to understand technical means of the present disclosure more clearly, and in order to make the above and other objects, features, and advantages of the present disclosure more obvious and understandable, specific embodiments of the present disclosure are set forth below.

### BRIEF DESCRIPTION OF DRAWINGS

In order to describe technical schemes in embodiments of the present disclosure more clearly, the drawings to be used in describing the embodiments will be introduced below in brief. Apparently, the drawings described below are some of the embodiments of the present disclosure, and those of ordinary skills in the art may also obtain other drawings according to these drawings without using any inventive effort. In the drawings:

FIG. 1 shows a schematic diagram of a locking nucleic acid-modified molecular barcode used in a gene rearrangement detection process provided by an embodiment of the present disclosure;

FIG. 2 shows a schematic diagram of non-specific amplification provided by an embodiment of the present disclosure;

FIG. 3 illustrates a flowchart of a method for inhibiting non-specific amplification in gene rearrangement detection provided by an embodiment of the present disclosure;

FIG. 4 shows a detailed flowchart of another method for inhibiting non-specific amplification in gene rearrangement detection provided by an embodiment of the present disclosure.

### DETAILED DESCRIPTION

Exemplary implementations of the present disclosure will be described in more detail below with reference to accompanying drawings. Although exemplary implementations of the present disclosure are shown in the drawings, it should be understood that the present disclosure may be implemented in various forms and should not be limited by the embodiments set forth herein. On the contrary, these embodiments are provided for a more thorough understanding of the present disclosure and for fully conveying the scope of the present disclosure to those skilled in the art.

The creative thinking of the present disclosure is that the traditional lymphoma diagnosis methods mainly comprise histopathology, immunology, etc., but the detection rate is low and it is easy to misjudge. Immunoglobulin gene rearrangement detection can assist lymphoma diagnosis and improve diagnostic accuracy, and the detection rate and diagnostic accuracy of lymphoma both are over 90% by gene rearrangement detection of heavy chain and light chain genes.

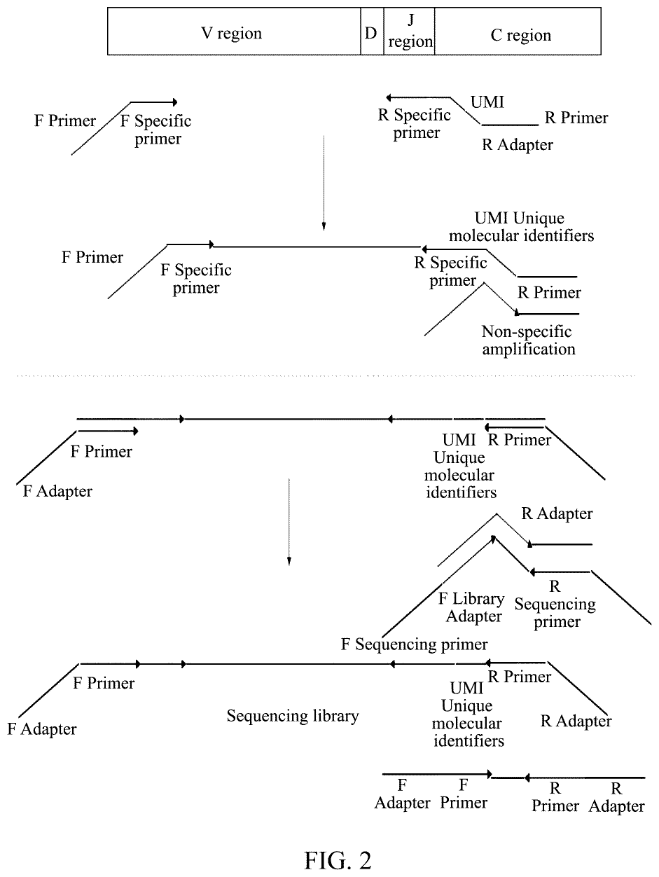

In gene rearrangement detection, PCR-high-throughput sequencing method has high resolution and can determine the specific sequence of each clone at the same time, so the detection accuracy is high. However, PCR-high-throughput sequencing relies on multiplex PCR to construct the library, and the amplification bias resulted in the library process greatly interferes with the detection results, which will lead to diagnostic errors in partial samples. Although the bias caused by PCR amplification reaction can be eliminated by molecular barcode sequencing technology at present, and the sequencing uniformity and sensitivity can be improved, as shown in FIG. 2, since molecular barcode sequences of forward primers and reverse primers bind to each other and will be amplified, the construction of high-throughput sequencing library based on molecular barcode technology will produce adapter self-connected fragments, i.e. non-specific amplification products, which will greatly interfere with the results of subsequent high-throughput sequencing.

In one embodiment of the present disclosure, a locked nucleic acid-modified molecular barcode is provided for inhibiting non-specific amplification in the process of gene rearrangement detection, the total number of bases in the molecular barcode, Q, is greater than or equal to 10, the molecular barcode has m locked nucleic acid modified sites, which divide the molecular barcode into m+1 segments, wherein the number of bases in each segment is 2 to 4, and m is greater than or equal to 2.

In some embodiments of the present disclosure, the bases of the molecular barcode may be base A, base T, base C, base G, degenerate base R, degenerate base Y, degenerate base M, degenerate base K, degenerate base S, degenerate base W, degenerate base H, degenerate base B, degenerate base V, degenerate base D, and degenerate base N.

In some embodiments of the present disclosure, the possible distribution among the total number of bases in the molecular barcode, the number of degenerate bases, and the number of locked nucleic acid modified sites is as follows:
1. the total number of bases in the molecular barcode, Q, is 10, the number of degenerate bases N is 4 or 5 or 6 or 7 or 8, the number of the locked nucleic acid-modified sites is 2, and the rest bases can be degenerate bases R, degenerate bases Y, degenerate bases M, degenerate bases K, degenerate bases S, degenerate bases W, degenerate bases H, degenerate bases B, degenerate bases V or degenerate bases D;
2. the total number of bases in the molecular barcode, Q, is 12, the number of degenerate bases N is 4 or 5 or 6 or 7 or 8 or 9, the number of the locked nucleic acid-modified sites is 4 or 3, and the rest bases can be degenerate bases R, degenerate bases Y, degenerate bases M, degenerate bases K, degenerate bases S, degenerate bases W, degenerate bases H, degenerate bases B, degenerate bases V or degenerate bases D;
3. the total number of bases in the molecular barcode, Q, is 14, the number of degenerate bases N is 8 or 9 or 10, the number of the locked nucleic acid-modified sites is 4, and the rest bases can be degenerate bases R, degenerate bases Y, degenerate bases M, degenerate bases K, degenerate bases S, degenerate bases W, degenerate bases H, degenerate bases B, degenerate bases V or degenerate bases D;
4. the total number of bases in the molecular barcode, Q, is 15, the number of degenerate bases N is 8 or 9 or 10, the number of the locked nucleic acid-modified sites is 5 or 4, and the rest bases can be degenerate bases R, degenerate bases Y, degenerate bases M, degenerate bases K, degenerate bases S, degenerate bases W, degenerate bases H, degenerate bases B, degenerate bases V or degenerate bases D;
5. the total number of bases in the molecular barcode, Q, is 18, the number of corresponding degenerate bases N is 8 or 9 or 10, the number of the locked nucleic acid-modified sites is 6 or 5, and the remaining bases can be degenerate bases R, degenerate bases Y, degenerate bases M, degenerate bases K, degenerate bases S, degenerate bases W, degenerate bases H, degenerate bases B, degenerate bases V or degenerate bases D.

The number of bases in each segment of molecular barcode modified by the locked nucleic acid is controlled to be 2 to 4. On one hand, since each segment modified by the locked nucleic acid needs to maintain the number of degenerate bases, so that the types of random binding between the molecular barcode and the target gene are sufficient, thereby the number of products is sufficient. Therefore, if the number of bases in a certain segment is too small, the number of types of random binding between amplification primers will be insufficient, thus it is difficult to maintain enough number of products, which will lead to the locked nucleic acid-modified sites not satisfying the requirements. On the other hand, when the number of bases in each segment modified the locked nucleic acid exceeds 4, the primers where the segment sequence is located may be directly bind to complementary primers, which results in non-specific amplification products.

From the stage of amplification reaction, because the molecular barcode can inhibit the non-specific amplification between the forward primers and the reverse primers, it can promote the full amplification of the forward primers and the reverse primers with the target genes respectively, whereby improving the efficiency of amplification reaction.

In some alternative embodiments, m satisfies:

[Q/4]≤m≤[Q/3],
in the formula, Q denotes the total number of bases in the molecular barcode, [Q/3] denotes the maximum integer not exceeding Q/3, and [Q/4] denotes the maximum integer not exceeding Q/4

In the technical schemes provided by some embodiments of the present disclosure, by controlling the relationship between the locked nucleic acid-modified sites and the total number of bases in the molecular barcode, and controlling m to satisfy the relationship of [Q/4]≤m≤[Q/3], the locked nucleic acid-modified molecular barcode can effectively inhibit the non-specific amplification between the forward primer and the reverse primer. Specifically, if the value of m is too small, the number of bases in a certain segment in the molecular bar code will be too much, whereby enhancing the binding ability of primers containing the molecular barcode to the fragments and regions of non-target genes to a certain extent, making the forward primers and reverse primers directly bind each other, which increases the number of non-specific amplification products to a certain extent; if the value of m is too large, the number of bases in a certain segment in the molecular barcode will be too small, and the number of types of unique molecular identifiers will be too small to a certain extent, which results that the locked nucleic acid-modified sites not satisfying the requirements to a certain extent.

In some alternative embodiments, the number of degenerate bases N is greater than or equal to 8.

In the technical schemes provided by some embodiments of the present disclosure, by controlling the number of degenerate bases N in the molecular barcode to be greater than or equal to 8, the sufficient number of degenerate bases N can be satisfied, and at the same time, more than two locked nucleic acid-modified sites can be made. Because the degenerate bases N represent any one of bases A, C, T and G, so that the types of molecular barcodes that can be selected are sufficient, whereby ways of random binding of the molecular barcode to the target gene are sufficient, and the function of molecular barcodes is normal.

In some alternative embodiments, the number of degenerate bases Nis 8 to 10.

In the technical schemes provided by some embodiments of the present disclosure, by controlling the number of degenerate bases N, the number of types of random binding between the molecular barcode and the target gene is sufficient, the number of products is sufficient, and the risk of non-specific amplification of the molecular barcode and the target gene can be reduced. Specifically, if the number value of degenerate base N is too small, the types of random binding between the molecular barcode and the target gene is insufficient, which may not satisfy the requirement of product number. If the number value of degenerate base N is too large, on one hand, the sequence length of the molecular barcode is too long, and the excessively long molecular barcode will increase the risk of DNA mismatch between the primers where the molecular barcode is located and the target gene to a certain extent, which will increase the number of non-specific amplification products. On the other hand, once the number of degenerate base N is increased, the locked nucleic acid-modified sites may not satisfy the requirement that the number of bases in each segment of the molecular barcode is 2 to 4. The number of bases in a certain segment in the molecular barcode may exceed 4, which will lead to direct binding between the primers where the molecular barcode is located and complementary primers, which results in non-specific amplification products.

In some alternative embodiments, the number of bases in at least two segments in the molecular barcode is same.

In some alternative embodiments, the number of bases in two adjacent segments in the molecular barcode is the same.

In the technical schemes provided by some embodiments of the present disclosure, the number of bases in two segments in the molecular barcode is controlled to be the same, or the number of bases in two adjacent segments is controlled to be the same, so that at least two segments in the molecular barcode are uniformly separated, and the uniformly separated molecular barcodes can be stably binded to the target gene in the complementary pairing process with the target gene, so as to enhance the binding degree between the molecular barcode and the target gene, avoid the direct binding between the primer where the molecular barcode is located and the complementary primer, and inhibit the self-connection of the adapter between the forward primer and the reverse primer.

In some alternative embodiments, the total number Q of bases in the molecular barcode is 10, and the locked nucleic acid-modified sites include a position k site and a position k+4 site in the direction from the 5′ end to the 3′ end of the molecular barcode, wherein k may be 3 or 4, and the corresponding locked nucleic acid modified-sites may be a position 3 site and a position 7 site, as well as a position 4 site and a position 8 site.

In the technical schemes provided by some embodiments of the present disclosure, the total number of bases in the molecular barcode is controlled to be 10, and the locked nucleic acid-modified sites comprise two sites, and the number of interval bases between the two sites is controlled to be 4, so that the sequences in the molecular barcode are kept a certain interval, and at the same time, the molecular barcode of 10 bases is roughly evenly separated, and the evenly separated molecular barcode can stably bind to the target gene in the complementary pairing process with the target gene, thus avoiding the direct mutual binding between the primer where the molecular barcode is located and the complementary primer, and inhibiting the self-connection of the adapters between the forward primer and the reverse primer.

It should be noted that the bases at these locked nucleic acid-modified sites can be specifically selected from base A, base T, base C and base G, and the bases at specific locked nucleic acid-modified sites need to be determined by complementary primers of the primers where the molecular barcodes are located.

When the locked nucleic acid-modified sites are a position 4 site and a position 8 site, the base at the position 4 site or the position 8 site can be specifically selected from base A, base T, base C and base G, that is to say, the base at the position 4 site or the position 8 site can be base A, base T, base C and base G, and the specific selection needs to be determined by the complementary primers of the primers where the molecular barcodes are located. When the base selected at the position 4 site is base A and the base at the position 8 site is T, the base sequence in the molecular barcode obtained is set forth in SEQ ID NO. 1.

Similarly, when the base selected at a position 3 site is base C and the base selected at a position 7 site is base A, the base sequence in the molecular barcode obtained is set forth in SEQ ID NO. 2.

In one embodiment of the present disclosure, a reverse primer is provided, which comprises, but is not limited to, an adapter primer, a locked nucleic acid-modified molecular barcode, and a specific primer.

In the technical schemes provided by some embodiments of the present disclosure, the primer where the molecular barcode is located is controlled, because there are less regions in the reverse primer which bind to the target gene than that in the forward primer, the reverse primer is designed and the binding degree between the reverse primer and the target gene can be improved through the molecular barcode in the designed reverse primer, thereby inhibiting the non-specific amplification between the forward primer and the reverse primer in the amplification primer.

In one embodiment of the present disclosure, a primer set is provided, the primer set comprising:
forward primer, and
reverse primer;
the forward primer comprises but are not limited to at least one of a FR1 forward primer, a FR2 forward primer, and a FR3 forward primer, wherein the forward primer may be the FR1 forward primer, the FR2 forward primer, the FR3 forward primer, also may be mixed forward primers of the FR1 forward primer and the FR2 forward primer, also may be mixed forward primers of the FR2 forward primer and the FR3 forward primer, also may be mixed forward primers of the FR1 forward primer and the FR3 forward primer, or also may be mixed forward primer of the FR1 forward primer, the FR2 forward primer and the FR3 forward primer.

In one embodiment of the present disclosure, a kit is provided, the kit comprises, but is not limited to, a reverse primer, a DNA polymerase, buffer, and magnetic bead for purification.

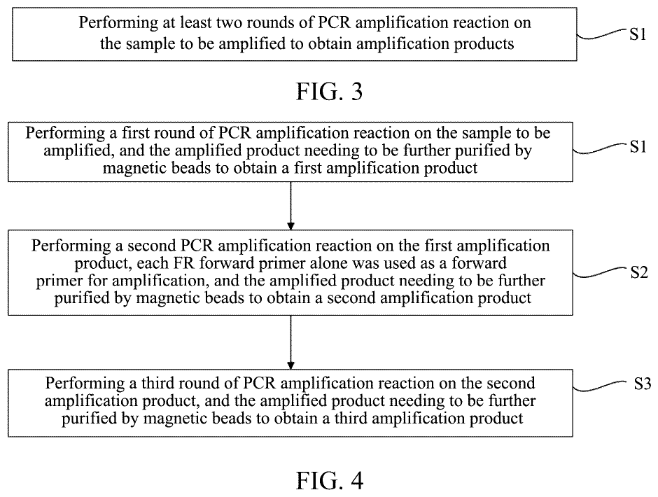

In one embodiment of the present disclosure, as shown in FIG. 3, a method for inhibiting non-specific amplification in gene detection is provided, comprising:
S1. performing at least two rounds of PCR amplification reaction on the sample to be amplified to obtain amplification products;
wherein, the primer used in a first round of PCR amplification reaction comprises the reverse primer, and the primer used in a second round of PCR amplification reaction comprises at least one of the FR1 forward primer, the FR2 forward primer and the FR3 forward primer.

In the technical schemes provided by some embodiments of the present disclosure, the first round of PCR amplification reaction is required to carry out and then the second round of PCR amplification reaction in the first two rounds of PCR amplification reaction. The primers used in the first round of PCR amplification reaction are reverse primers with the locked nucleic acid-modified molecular barcodes, so that the reverse primers with the molecular barcodes can be amplified with target genes, which causes products of the first round of amplification all with a molecular barcode. And the products of the first round of PCR amplification can be further amplified by carrying out the second round of PCR amplification with the forward primers, so that products of the first round of PCR amplification can be further amplified, which promotes products of the first round of PCR to be further amplified with the forward primers. Therefore, the total amount of amplification products is sufficient, which is convenient for subsequent gene rearrangement detection.

In some alternative embodiments, in at least two rounds of the PCR amplification reaction, the primer used in the last round of the PCR amplification reaction is an adapter primer.

In the technical schemes provided by some embodiments of the present disclosure, the primer used for controlling the last round of PCR amplification reaction is an adapter primer, and the adapter primer is used to complete the DNA fragment of the whole amplification product, which is convenient for subsequent gene rearrangement detection.

It should be noted that, in general, after two rounds of PCR amplification reaction are carried out, the last round of PCR amplification reaction can be carried out. By adopting at least two rounds of PCR amplification reaction above, the direct introduction of the primer set comprising the forward primer and the reverse primer can be avoided, because the positive primer and the reaction primer in the primer set can be directly bound to each other in the PCR amplification reaction stage. Therefore, by adopting different rounds of PCR amplification reaction, the reverse primer and the forward primer are respectively subjected to PCR amplification reaction, which can inhibit the non-specific amplification between the forward primer and the reverse primer and reduce the number of non-specific amplification products.

In some alternative embodiments, the concentration of the reverse primer is from 9 μmol/L to 11 μmol/L, and the added amount of the reverse primer is from 5 μL to 7 μL.

In the technical schemes provided by some embodiments of the present disclosure, the concentration of the reverse primers and the added amount of the reverse primers are generally controlled, such that the reverse primers can sufficiently amplify the sample to be amplified in the first round of PCR amplification reaction. Therefore, the number of the first amplification products is sufficient to provide sufficient templates for the second round of PCR amplification reaction.

In order to better facilitate the use of amplification reaction system, it is necessary to control the added amount of primers, and after the added amount of the reverse primers is controlled, the concentration of the reverse primers should also be controlled to obtain a sufficient number of the first amplification products.

In some alternative embodiments, the total weight of DNA in the sample to be amplified is greater than or equal to 100 ng.

In the technical schemes provided by some embodiments of the present disclosure, the total weight of DNA in the sample to be amplified is controlled so that the number of DNA fragments is sufficient, and enough DNA fragments can make the number of amplification products sufficient. Therefore, controlling the total weight of DNA can provide sufficient products for the first round of PCR amplification reaction and the second round of PCR amplification reaction, making the final amount of amplification products sufficient, to facilitate subsequent gene rearrangement detection.

In one embodiment of the present disclosure, a use of the locked nucleic acid-modified molecular barcode in preparing a reagent for gene rearrangement detection is provided, including, but not limited to, the molecular barcode being used in preparing a forward primer or probe for gene rearrangement detection.

### Example 1

Example 1 provided a locked nucleic acid-modified molecular barcode, the number of bases in the molecular barcode was 10, and the locked nucleic acid-modified sites were a position 4 site and a position 8 site, wherein the base at the position 4 site was base A and the base at the position 8 site was base T, and the sequence of the locked nucleic acid-modified molecular barcode was 5′-NNA*NNNT*NN-3′ set forth in SEQ ID NO. 1, wherein * denoted the locked nucleic acid (LNA)-modified site.

### Example 2

Example 2 provided a reverse primer, which, on the basis of Example 1, comprised an adapter primer, a locked nucleic acid-modified molecular barcode, and a specific primer, the specific sequence of which was set forth in SEQ ID NO. 3.

### Example 3

Example 3 provided a primer set comprising a forward primer and a reverse primer of Example 2, wherein the forward primer comprised a FR1 forward primer, a FR2 forward primer, or a FR3 forward primer, the specific primer set being shown in Table 1.

TABLE 1
Sequence listing of primer set

| Targeting region | Sequence direction (5′→3′) |
|---|---|
| J Reverse Primer | GACTGGAGTTCAGACGTGTGCTCTTCCGATCNNNA*NNNT*NNCTT |
| Modified by | ACCTGAGGAGACGGTGACC (SEQ ID NO. 3) |
| Locked Nucleic | |
| Acid | |
| J Reverse Primer | GACTGGAGTTCAGACGTGTGCTCTTCCGATCNNC*NNNA*NNNCTT |
| Modified by | ACCTGAGGAGACGGTGACC (SEQ ID NO. 4) |
| Locked Nucleic | |
| Acid | |
| FR1 Forward | ACACTCTTTCCCTACACGACGCTCTTCCGATCGGCCTCAGGAAGGT |
| Primer | CTCCTGCAAG (SEQ ID NO. 5) |
| | ACACTCTTTCCCTACACGACGCTCTTCCGATCGTCTGGTCCTACGCT |
| | GGAACCC (SEQ ID NO. 6) |
| | ACACTCTTTCCCTACACGACGCTCTTCCGATCCTGGGGGTCCCTGA |
| | GACTCTCCTG (SEQ ID NO. 7) |
| | ACACTCTTTCCCTACACGACGCTCTTCCGATCCTTCGGAGACCCCTG |
| | TCCCTCACCTG (SEQ ID NO.8) |
| | ACACTCTTTCCCTACACGACGCTCTTCCGGGGAGTCTCTGAAGATC |
| | TCCTGT (SEQ ID NO. 9) |
| | ACACTCTTTCCCTACACGACGCTCTTCCGATTCGCAGACCCTCTCAC |
| | TCACCTGTG (SEQ ID NO. 10) |
| FR2 Forward | ACACTCTTTCCCTACACGACGCTCTTCCGATCTGTCAGCCCCCAGG |
| Primer | GAAGG (SEQ ID NO. 11) |
| | ACACTCTTTCCCTACACGACGCTCTTCCGATCGGTCCGCCCAGGCT |
| | CCAGGGAA (SEQ ID NO. 12) |
| | ACACTCTTTCCCTACACGACGCTCTTCCGATCTGCCCCCCCCCAGG |
| | GAAGG (SEQ ID NO. 13) |
| | ACACTCTTTCCCTACACGACGCTCTTCCGATCGGGTGGCCCAGATG |
| | CCCGGGAAGG (SEQ ID NO. 14) |
| | ACACTCTTTCCCTACACGACGCTCTTCCGATCTGGATCAGGCAGTC |
| | CCCCATCGAGAG (SEQ ID NO. 15) |
| | ACACTCTTTCCCTACACGACGCTCTTCCGATCTTGGGGGCGACAGG |
| | CCCCTGGACAA (SEQ ID NO. 16) |
| FR3 Forward | ACACTCTTTCCCTACACGACGCTCTTCCGATCTGGAGCTGAGCAGC |
| Primer | CTGAGATCTGA (SEQ ID NO. 17) |
| | ACACTCTTTCCCTACACGACGCTCTTCCGATCCATGACCAACATGG |
| | ACCCCTGTGGA (SEQ ID NO. 18) |
| | ACACTCTTTCCCTACACGACGCTCTTCCGATCTCTGCAAATGAACA |
| | GCCTGAGAGCC (SEQ ID NO. 19) |
| | ACACTCTTTCCCTACACGACGCTCTTCCGATCGAGCTCTGTGGACC |
| | GCCGGGACACG (SEQ ID NO. 20) |
| | ACACTCTTTCCCTACACGACGCTCTTCCGATCCAGCACCGCCTACCT |
| | GCAGTGGAGC (SEQ ID NO. 21) |
| | ACACTCTTTCCCTACACGACGCTCTTCCGATCGTTCTCCCTGCAGCT |
| | GAACTCTGTG (SEQ ID NO. 22) |
| | ACACTCTTTCCCTACACGACGCTCTTCCGATCCCAGCACGGCATAT |
| | CTGCAGATCAG (SEQ ID NO. 23) |
| J Reverse Primer | GACTGGAGTTCAGACGTGGCTCTTCCGATCNNNNNNNNCTTACCTG |
| Not Modified by | AGGAGACGGGACC (SEQ ID NO. 24) |
| locked Nucleic | |
| Acid | |
| Illumina-R | GACTGGAGTTCAGACGTGGCTCTTCCGATC (SEQ ID NO. 25) |
| Adapter Primer | |

Note:
* represented the locked nucleic acid (LNA)-modified sites.

### Example 4

Example 4 provided a kit comprising the reverse primer of Example 2, DNA polymerase, buffer, and magnetic bead.

### Example 5

Example 5 provided a method for inhibiting nonspecific amplification in gene rearrangement detection, as shown in FIG. 4, comprising:
S1. Performing a first round of PCR amplification reaction on the sample to be amplified. The specific amplification reaction system was shown in Table 2. The amplification reaction procedure was shown in Table 3. And the amplification product needed to be further purified by magnetic beads to obtain a first amplification product; wherein, the volume ratio of the magnetic beads to the amplification reaction system in magnetic bead purification was 0.8;
S2. Performing a second PCR amplification reaction on the first amplification product. The specific amplification reaction system was shown in Table 4, and the amplification reaction program was shown in Table 5. Each FR forward primer alone was used as a forward primer for amplification. And the amplification product needed to be further purified by magnetic beads to obtain a second amplification product; wherein, the volume ratio of the magnetic beads to the amplification reaction system in magnetic bead purification was 0.6;
S3. Performing a third round of PCR amplification reaction on the second amplification product. The specific amplification reaction system was shown in Table 6. The amplification reaction program was shown in Table 7. And the amplification product needed to be further purified by magnetic bead to obtain a third amplification product; wherein, the volume ratio of the magnetic beads to the amplification reaction system in magnetic bead purification was 0.8;
the primer used in the first PCR amplification reaction was the reverse primer set forth in SEQ ID NO. 2, the primer used in the second PCR amplification reaction was the FR1 forward primer, the FR2 forward primer and the FR3 forward primer, and the primers used in the third PCR amplification reaction were Illumina-R adapter primers.

TABLE 2
Table of the first round of PCR amplification reaction system

| Composition | Volume | |
|---|---|---|
| 2*KOD-Multi & Epi Buffer | 25 | μL |
| Lymph node DNA | X μL (total DNA weight 100 ng) | |
| Reverse primer (10 μM) | 6 | μL |
| KOD-Multi & Epi | 1 | μL |
| DNase-Free & RNase-Free Water | (18-X) | μL |
| Total | 50 | μL |

Note:
in the table, 0 < X ≤ 18 μL.

TABLE 3
Table of the first round of PCR amplification reaction program

| | | | |
|---|---|---|---|
| Steps | Temperature | Time | Cycles |

| | | | | | |
|---|---|---|---|---|---|
| Predenaturation | 94° | C. | 2 | min | 1 |
| Denaturation | 98° | C. | 30 | s | |
| Annealing | 62° | C. | 30 | s | |
| Extension | 68° | C. | 1 | min | |
| Extension after | 68° | C. | 5 | min | |
| amplification | | | | | |

| | | | |
|---|---|---|---|
| Preservation | 4° | C. | ∞ |

TABLE 4
Table of the second round of PCR amplification reaction system

| Composition | Volume | |
|---|---|---|
| 2*KOD-Multi & Epi Buffer | 25 | μL |
| The first round of PCR amplification products | 12.5 | μL |
| FR1 forward primer, FR2 forward primer and | 6 | μL |
| FR3 forward primer (the concentration of each | | |
| forward primer was 10 μM) | | |
| Illumina-R adapter primer (10 μM) | 1.5 | μL |
| KOD-Multi & Epi | 1 | μL |
| DNase-Free & RNase-Free Water | 4 | μL |
| Total | 50 | μL |

TABLE 5
List of the second round of PCR amplification reaction program

| | | | |
|---|---|---|---|
| Steps | Temperature | Time | Cycle number |

| | | | | | |
|---|---|---|---|---|---|
| Predenaturation | 94° | C. | 2 | min | 1 |
| Denaturation | 98° | C. | 30 | s | 21-25 |
| Annealing | 62° | C. | 30 | s | |
| Extension | 68° | C. | 30 | s | |
| Extension after | 68° | C. | 5 | min | 1 |
| amplification | | | | | |

| | | | | |
|---|---|---|---|---|
| Preservation | 4° | C. | ∞ | 1 |

TABLE 6
Table of the third round of PCR amplification reaction system

| Composition | Volume |
|---|---|
| 2*KOD-Multi & Epi Buffer | 25 μL |
| The second PCR amplification product | 20 μL |
| Hieff NGS ® Stubby UDI Primer Kit for | 5 μL |
| Illumina Detection Kit | |
| Total | 50 μL |

TABLE 7
List of the third round of PCR amplification reaction program

| | | | |
|---|---|---|---|
| Steps | Temperature | Time | Cycle number |

| | | | | | |
|---|---|---|---|---|---|
| Predenaturation | 95° | C. | 3 | min | 1 |
| Denaturation | 98° | C. | 30 | s | 8-12 |
| Annealing 1 | 60° | C. | 30 | s | |
| Extension | 68° | C. | 30 | s | |
| Extension after | 68° | C. | 5 | min | 1 |
| amplification | | | | | |

| | | | | |
|---|---|---|---|---|
| Preservation | 4° | C. | ∞ | 1 |

### Example 6

Example 6 provided another locked nucleic acid-modified molecular barcode, the number of bases in the molecular barcode was 10, and the locked nucleic acid-modified sites were a position 3 site and a position 7 site, wherein the base at the position 3 site was base C and the base at the position 7 site was base A, and the sequence of the molecular barcode was 5′-NNC*NNNA*NNN-3′ set forth in SEQ ID NO. 2, wherein * denoted the locked nucleic acid (LNA)-modified site.

### Example 7

Example 7 provided a reverse primer, which, on the basis of Example 6, comprised an adapter primer, a locked nucleic acid-modified molecular barcode, and a specific primer, the specific sequence of which was set forth in SEQ ID NO. 4.

### Example 8

Example 8 provided a primer set comprising a forward primer and a reverse primer of Example 7, wherein the forward primer comprised a FR1 forward primer, a FR2 forward primer, or a FR3 forward primer, the specific primer set being shown in Table 1.

### Example 9

Example 9 provided another method for inhibiting non-specific amplification in gene rearrangement detection. For brief description, reference can be made to the corresponding contents in the preceding method examples for what not mentioned in the example; compared with Example 5, the only difference of Example 9 is that a reverse primer comprising the molecular barcode of Example 6 was used.

### Comparative Example 1

Based on the control principle, Comparative Example 1 provided a molecular barcode not modified by locked nucleic acid with a specific sequence of 5′-NNNNNNNNNN-3′.

The sequence of the reverse primer corresponding to the molecular barcode was shown in Table 1, and the other sequences and process conditions of the method were the same as those of the aforementioned Example 1.

### Related Tests

The PCR amplification products obtained in Example 5, Example 9 and Comparative Example 1 were subjected to gene rearrangement detection, and the gene rearrangement detection data of Example 5, Example 9 and Comparative Example 1 were compared, and the results were shown in Tables 8 and 9.

### Methods of Related Tests

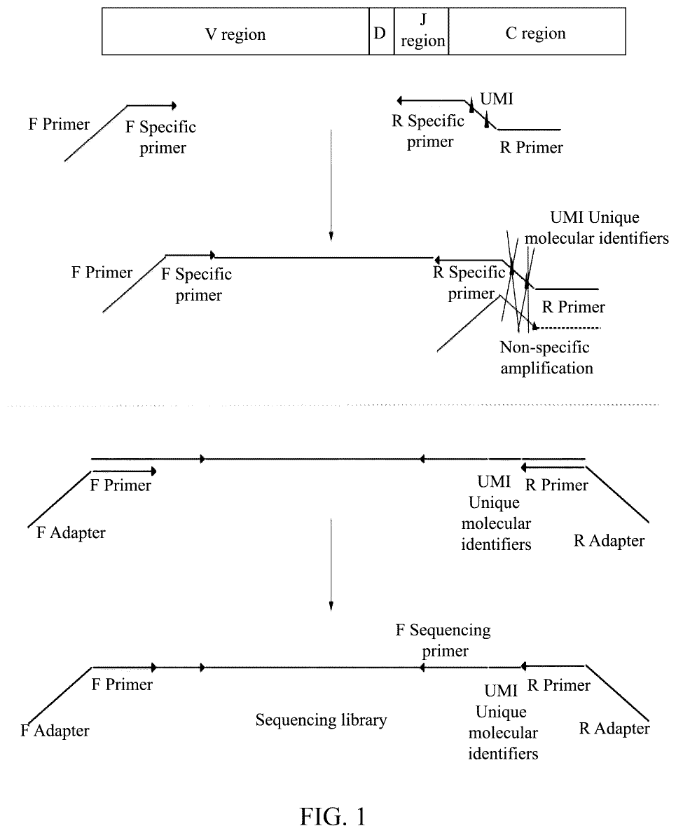

The reverse primers comprising a molecular barcode not modified by locked nucleic acid were sequentially added to PCR reaction tubes 1-3, the reverse primers comprising a locked nucleic acid-modified molecular barcode in Example 5 were sequentially added to PCR reaction tubes 4-6, the reverse primers comprising a locked nucleic acid-modified molecular barcode in Example 9 were sequentially added to PCR reaction tubes 7-9, and the forward primers in PCR reaction tubes 1, 4 and 7 were FR1 forward primers, the forward primers in PCR reaction tubes 2, 5 and 8 were FR2 forward primers, and the forward primers in PCR reaction tubes 3, 6 and 9 were FR3 forward primers, and the PCR amplification reactions were carried out respectively. For the amplification reaction systems and amplification procedures, see Tables 2 to 7. For the amplification methods, see Example 5 and Example 9. The amplification product was purified prior to being tested on an instrument as a sequencing library. The model of the testing instrument was Illumina NovaSeq, and the testing length of the instrument was PE150, and the testing data was read;
wherein, the principle of the locked nucleic acid-modified molecular barcode in the process of gene rearrangement detection was shown in FIG. 1.

TABLE 8
Comparative table of gene rearrangement detection data of Example 5 and Comparative Example 1

| Sample names | Tube 1 | Tube 2 | Tube 3 | Tube 4 | Tube 5 | Tube 6 |
|---|---|---|---|---|---|---|
| Sequencing label number | UDI | UDI | UDI | UDI | UDI | UDI |
| | primer 0001 | primer 0002 | primer 0003 | primer 0004 | primer 0005 | primer 0006 |
| Number of Total Reads | 2200082 | 1920135 | 2109326 | 2105642 | 1962453 | 2305349 |
| Number of Target Reads | 2160481 | 1 841409 | 1 993 313 | 2059840 | 1885892 | 2258995 |
| species | | | | | | |
| Ratio of valid Reads | 98.2% | 95.9% | 94.5% | 97.8% | 96.1% | 98.0% |
| Number of Reads with | 270060 | 114167 | 133552 | 1690060 | 1525669 | 1956922 |
| specific unique molecular | | | | | | |
| identifiers | | | | | | |
| Percentage of Reads with | 12.5% | 6.2% | 6.7% | 82.1% | 80.9% | 86.6% |
| specific unique molecular | | | | | | |
| identifiers | | | | | | |
| number of Reads with adapter | 1849372 | 1701462 | 1825874 | 196921 | 155963 | 178687 |
| self-connection | | | | | | |
| Percentage of number of | 85.6% | 92.4% | 91.6% | 9.56% | 8.27% | 7.91% |
| Reads with adapter | | | | | | |
| self-connection | | | | | | |

TABLE 9
Comparative table of gene rearrangement detection
data of Example 9 and Comparative Example 1

| Sample names | Tube 1 | Tube 2 | Tube 3 | Tube 7 | Tube 8 | Tube 9 |
|---|---|---|---|---|---|---|
| Percentage of | 85.6% | 92.4% | 91.6% | 10.26% | 9.16% | 8.83% |
| Number of | | | | | | |
| Reads with | | | | | | |
| adapter self- | | | | | | |
| connection | | | | | | |

It can be seen from Table 8 that, through the locked nucleic acid-modified molecular barcode set forth in SEQ ID NO. 1, during the construction of PCR-high-throughput sequencing library for gene rearrangement, the percentage of number of Reads with adapter self-connection achieved a decrease from 85.6%, 92.4% and 91.6% to 9.56%, 8.27% and 7.91%, respectively, which was significantly decreased by one order of magnitude.

However, through the locked nucleic acid-modified molecular barcode set forth in SEQ ID NO. 2, during the construction of PCR-high-throughput sequencing library for gene rearrangement, the percentage of number of Reads with adapter self-connection achieved a decrease from 85.6%, 92.4% and 91.6% to 10.26%, 9.16% and 8.83%, respectively, which was also significantly decreased by one order of magnitude.

It is shown that, the present disclosure could significantly decrease the number of non-specific amplification products in the gene rearrangement detection process by the molecular barcode with a specific number of locked nucleic acid modifications at specific positions.

By comparing the data in Table 8 and Table 9, it could be seen that, the percentage of number of reads with adapter self-connection for the molecular barcode modified by the locked nucleic acid as shown in SEQ ID NO. 1 was different from that for the molecular barcode modified by the locked nucleic acid set forth in SEQ ID NO. 2. The percentage of number of reads with adapter self-connection for the molecular barcode modified by the locked nucleic acid set forth in SEQ ID NO. 1 was 9.56%, 8.27% and 7.91%, respectively, while the percentage of number of reads with adapter self-connection for the molecular barcode modified by the locked nucleic acid set forth in SEQ ID NO. 2 was 10.26%, 9.16% and 8.83%, respectively. It is shown that, different positions and bases for the locked nucleic acid modifications have different abilities to inhibit adapter self-connection between the forward primers and the reverse primers.

“Include”, “comprise” or any other variations thereof occurred herein are intended to cover a non-exclusive inclusion, such that a process, method, article, or device that includes a series of elements includes not only the elements but also other elements which are not expressly listed, or further includes elements inherent to such a process, method, article, or device. An element defined by a statement “include . . . ” does not exclude presence of additional identical elements in the process, method, article or device that includes the element, without more limitations. It should be understood by those skilled in the art that throughout the specification, the terms used herein should be understood to have meanings as commonly used in the art unless otherwise specifically stated. Accordingly, all technical and scientific terms used herein have the same meanings as generally understood by those of skill in the art to which the present disclosure pertains, unless otherwise defined. In case of any contradiction, the specification takes precedence.

Unless otherwise specified, various raw materials, reagents, instruments and device and so on used in the present disclosure can be commercially purchased or prepared by existing methods.

The endpoints and any values of the ranges disclosed herein are not limited to the exact ranges or values, which should be understood as comprising values close to the ranges or values. For numerical ranges, combining endpoint values of the respective ranges with other endpoint values, endpoint values of the respective ranges with individual point values, and individual point values with other individual point values may obtain one or more new numerical ranges, which are to be considered specifically disclosed herein.

Although alternative embodiments of the present disclosure have been described, those skilled in the art may make additional changes and modifications to the embodiments once underlying inventive concepts are known. Therefore, the appended claims are intended to be interpreted to encompass alternative embodiments as well as all changes and modifications falling within the scope of the present disclosure.

Apparently, various modifications and variations may be made to the present disclosure by those skilled in the art without departing from the spirit and scope of the present disclosure. Thus, if these modifications and variations to the present disclosure are within the scope of the claims of the present disclosure and their equivalent techniques, the present disclosure is also intended to include these modifications and variations.

1. A locked nucleic acid-modified molecular barcode for inhibiting non-specific amplification in the process of gene rearrangement detection,
wherein,
the total number of bases in the molecular barcode, Q, is greater than or equal to 10, the molecular barcode has m locked nucleic acid-modified sites, which divide the molecular barcode into m+1 segments, wherein the number of bases in each segment is 2 to 4, and m is greater than or equal to 2.

2. The molecular barcode according to claim 1, wherein m satisfies:
[Q/4]≤m≤[Q/3],
in the formula, Q denotes the total number of bases in the molecular barcode, [Q/3] denotes a maximum integer not exceeding Q/3, and [Q/4] denotes a maximum integer not exceeding Q/4.

3. The molecular barcode according to claim 1, wherein, the number of degenerate bases N is greater than or equal to 8.

4. The molecular barcode according to claim 3, wherein, the number of the degenerate bases N is 8 to 10.

5. The molecular barcode according to claim 1, wherein, the number of bases in at least two segments in the molecular barcode is same.

6. The molecular barcode according to claim 5, wherein, the number of bases in two adjacent segments in the molecular barcode is same.

7. The molecular barcode according to claim 1, wherein, the total number of bases in the molecular barcode, Q, is 10, and the locked nucleic acid-modified sites comprise a position k site and a position k+4 site along the direction from the 5′ end to the 3′ end of the molecular barcode, wherein, k is greater than 2.

8. The molecular barcode according to claim 7, wherein, the locked nucleic acid-modified sites comprise a position 4 site and a position 8 site.

9. The molecular barcode according to claim 8, wherein, the sequence of the molecular barcode is set forth in SEQ ID NO. 1.

10. The molecular barcode according to claim 7, wherein, the locked nucleic acid-modified sites comprise a position 3 site and a position 7 site.

11. The molecular barcode according to claim 10, wherein, the sequence of the molecular barcode is set forth in SEQ ID NO. 2.

12. A reverse primer, wherein, the reverse primer comprises an adapter primer, a molecular barcode, and a specific primer, and the molecular barcode is a locked nucleic acid-modified molecular barcode according to claim 1.

13. The reverse primer according to claim 12, wherein, the sequence of the reverse primer is set forth in SEQ ID NO. 3; or
the sequence of the reverse primer is set forth in SEQ ID NO. 4.

14. A primer set, wherein, the primer set comprises:
forward primer, and
the reverse primer according to claim 12;
the forward primer comprises at least one of FR1 forward primer, FR2 forward primer and FR3 forward primer.

15. A kit, wherein, the kit comprises:
DNA polymerase, buffer, and
the reverse primer according to claim 12.

16. A method for inhibiting non-specific amplification in gene rearrangement detection, which use the primer set according to claim 14 to perform an amplification reaction, the method comprising:
performing at least two rounds of PCR amplification reaction on the sample to be amplified to obtain an amplification product;
wherein, the primer used in a first round of PCR amplification reaction comprises the reverse primer, and the primer used in a second round of PCR amplification reaction comprises at least one of the FR1 forward primer, the FR2 forward primer and the FR3 forward primer.

17. The method according to claim 16, wherein, in the at least two rounds of PCR amplification reaction, the primer used in the last round of PCR amplification reaction is an adapter primer.

18. The method according to claim 16, wherein, the concentration of the reverse primer is from 9 μmol/L to 11 μmol/L, and the added amount of the reverse primer is from 5 μL to 7 μL.

19. The method according to claim 16, wherein, the total weight of DNA in the sample to be amplified is greater than or equal to 100 ng.

20. Use of a locked nucleic acid-modified molecular barcode according to claim 1 in preparing a reagent for gene rearrangement detection.
